(19) United States
(12) Patent Application Publication
(10) Pub. No.: US 2010/0160264 A1
(43) Pub. Date: Jun. 24, 2010

(54) CRYSTALLINE SOLID FORMS

(76) Inventors: Josef Wieser, Polling (AT); Arthur Pichler, Jenbach (AT); Andreas Hotter, Worgl (AT)

Correspondence Address: Manelii Denison & Selter PLLC, 2000 M Street, 7th Floor, Washington DC, DC, 20036, US

(21) Appl. No.: 12/665,330

(22) PCT Filed: Jun. 20, 2008
(86) PCT No.: PCT/EP2008/057848

§ 371 Date: Feb. 16, 2010

(30) Foreign Application Priority Data
Jun. 21, 2007 (EP) 07110773.4
Nov. 14, 2007 (EP) 07120674.2

(51) Int. Cl. A61K 31/65; C07C 237/26; A61P 31/00

(52) U.S. Cl. 514/152; 552/205

(57) ABSTRACT

The present invention relates to the new crystalline solid form VI and VIII of tigecycline and processes for the production of form VI and VIII. It further relates to a new method for preparing Form I and Form III in high polymorphic purity.

### FIELD OF THE INVENTION

The present invention relates to the new crystalline solid forms VI and VIII of tigecycline and processes for the production of forms VI and VIII. It further relates to a new method for preparing form I and form III in high polymorphic purity.

### BACKGROUND OF THE INVENTION

Tigecycline, (4S,4aS,5aR,12aS)-4,7-Bis(dimethylamino)-9-[[2-[(1,1-dimethylethyl)amino]acetyl]amino]-1,4,4a,5,5a,6,11,12a-octahydro-3,10,12,12a-tetrahydroxy-1,11-dioxo-2-naphthacene carboxamide (Form. 1), is a 9-t-butylglycylamido derivative of minocycline (Merck Index 14^{th} Edition, monograph number 9432, CAS Registry Number 220620-09-7). Compared to other tetracycline antibiotics tigecycline is more active against tetracycline-resistant strains and also more tolerable. Tigecycline possesses activity against bacterial isolates containing the two major determinants responsible for tetracycline-resistance: ribosomal protection and active efflux of drug out of the bacterial cell. Further tigecycline has broad spectrum activity as it is active against gram-positive pathogens (e.g. methicillin-resistant Staphylococcus aureus, vancomycin-resistant Enterococci), gram-negative pathogens (e.g. Acinetobacter baumannii, Stenotrophomonas maltophilia) and anaerobic pathogens. It is used for the treatment of complicated skin and skin structure infections and intra-abdominal infections (P. J. Petersen et al., Antimicrob. Agents and Chemoth. 43:738-744 (1999); R. Patel et al., Diagnostic Microbiology and Infectious Disease 38:177-179 (2000); H. W. Boucher et al., Antimicrob. Agents and Chemoth. 44:2225-2229 (2000); D. J. Biedenbach et al., Diagnostic Microbiology and Infectious Disease 40:173-177 (2001); P. J. Petersen et al., Antimicrob. Agents and Chemoth. 46:2595-2601 (2002); D. Milatovic et al., Antimicrob. Agents and Chemoth. 47:400-404 (2003); T. Hirata et al., Antimicrob. Agents and Chemoth. 48:2179-2184 (2004); G. A. Pankey Journal of Antimicrobial Chemotherapy 56, 470-480 (2005); R. Harris et al., P&T 31:18-59 (2006)).

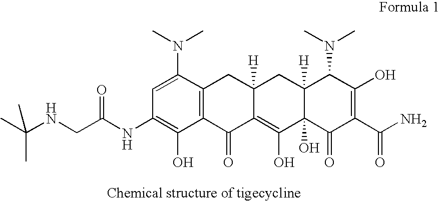

Tigecycline is only available as injectable antibiotic as its oral bioavailability is very limited. The orange lyophilized powder or cake is available in 5 ml vials containing 50 mg of the amorphous agent. (R. Harris et al., P&T 31:18-59 (2006)) The lyophilization process needs special conditions like low temperatures and low oxygen atmosphere. This process is quite expensive because of special equipment and handling (WO 2006/128150 A2). That's why new crystalline polymorphic forms of tigecycline are of interest because there is no need for lyophilization as crystalline forms are more stable than amorphous forms.

Patent application WO 2006/128150 discloses crystalline forms I to V of Tigecycline and methods for their preparation. Nevertheless, there remains a need for alternative polymorphic forms of Tigecycline which have properties suitable for pharmaceutical processing on a commercial scale.

### SUMMARY OF THE INVENTION

The present invention refers to crystalline form VIII of Tigecycline.

Crystalline form VIII of Tigecycline can be described by an X-ray powder diffraction pattern with peaks at 2-theta angles of 5.1°±0.2°, 9.1°±0.2°, 10.4°±0.2°, 12.8°±0.2°, 13.8°±0.2°, 14.9°±0.2°, 15.4°±0.2°, 16.4°±0.2°, 17.1°±0.2°, 18.6°±0.2°, 20.4°±0.2°, 21.8°±0.2°, 24.2°±0.2° and 25.8°±0.2°. A characteristic X-ray powder diffraction pattern of form VIII of Tigecycline is shown in FIG. 1 and some characteristic peaks are listed in Table 1.

Alternatively crystalline form VIII of Tigecycline can be described by an infrared spectrum comprising peaks at 3381±2 cm⁻¹, 3231±2 cm⁻¹, 2953±2 cm⁻¹, 1716±2 cm⁻¹, 1695±2 cm⁻¹, 1520±2 cm⁻¹, 1415±2 cm⁻¹, 1210±2 cm⁻¹, 1073±2 cm⁻¹, 870±2 cm⁻¹, 693±2 cm⁻¹, and 659±2 cm⁻¹.

In a further aspect the invention is related to a process for the preparation of crystalline form VIII of Tigecycline comprising the steps of:
a) slurrying Tigecycline in acetone at 30° C. or below;
b) stirring the slurry at 30° C. or below to effect transformation of the suspended form into form VIII; and
c) isolating crystalline form VIII of Tigecycline;

In addition the present invention provides another modification of the process of preparing form VIII of Tigecycline comprising the steps of:
a) slurrying Tigecycline in acetone at 30° C. or below;
b) heating the suspension to 56° C. or below under stirring to obtain a clear solution;
c) slowly cooling down the solution to 25 to 0° C. to effect crystallization; and
d) isolating crystalline form VIII of Tigecycline.

The present invention also relates to form VIII of Tigecycline for use as a medicament. In another aspect the present invention relates to the use of form VIII of Tigecycline for the preparation of a medicament for the treatment of infections.

In another embodiment, the present invention relates to form VI of tigecycline characterized by an X-ray powder diffraction pattern with peaks at 6.3, 6.7, 8.9, 9.4, 9.7, 12.1, 12.5, 13.7, 17.0, 17.8, 18.0, 18.5, 20.1, 21.5, 22.6, 23.3, 23.8 and 24.6 degrees 2 theta.

The present invention also provides a process for preparing form VI of Tigecycline comprising the steps of:

a) dissolving Tigecycline in a suitable solvent
b) stirring the solution at room temperature or below to effect crystallization
c) optionally isolating crystalline form VI of Tigecycline

The present invention also provides a process for preparing form VI of Tigecycline characterized in that a suspension of Tigecycline in a suitable solvent is seeded with crystals of form VI and the suspension is stirred at a suitable temperature in order to effect transformation of the used form of Tigecycline into form VI.

In another aspect the invention provides a new process for preparing form I comprising the steps of:

a) dissolving Tigecycline in ethanol,
b) stirring the solution at room temperature or below to effect crystallization
c) isolating crystalline form I of Tigecycline

In another aspect the invention provides a new process for preparing form III in essentially pure polymorphic form comprising the steps of:

a) dissolving Tigecycline in methyl ethyl ketone,
b) stirring the solution at room temperature or below to effect crystallization
c) isolating crystalline form III of Tigecycline

The present invention also relates to form VI of Tigecycline for use as a medicament.

In another aspect the present invention relates to the use of form VI of Tigecycline for the preparation of a medicament for the treatment of infections.

Other objects, features, advantages and aspects of the present invention will become apparent to those of skill from the following description. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and from reading the other parts of the present disclosure.

### BRIEF DESCRIPTION OF THE DRAWINGS

FIG. 1: X-ray powder diffraction pattern of form VIII of Tigecycline

FIG. 2: Infrared spectrum of form VIII of Tigecycline

FIG. 3: Differential scanning calorimetric curve of form VIII of Tigecycline

FIG. 4: Thermogravimetric analysis curve of form VIII of Tigecycline

FIG. 5: X-ray powder diffraction pattern of form VI of Tigecycline

FIG. 6: Infrared spectrum of form VI of Tigecycline

FIG. 7: Differential scanning calorimetric curve of form VI of Tigecycline

FIG. 8: X-ray powder diffraction pattern of form I of Tigecycline

FIG. 9: X-ray powder diffraction pattern of essentially pure form III of Tigecycline

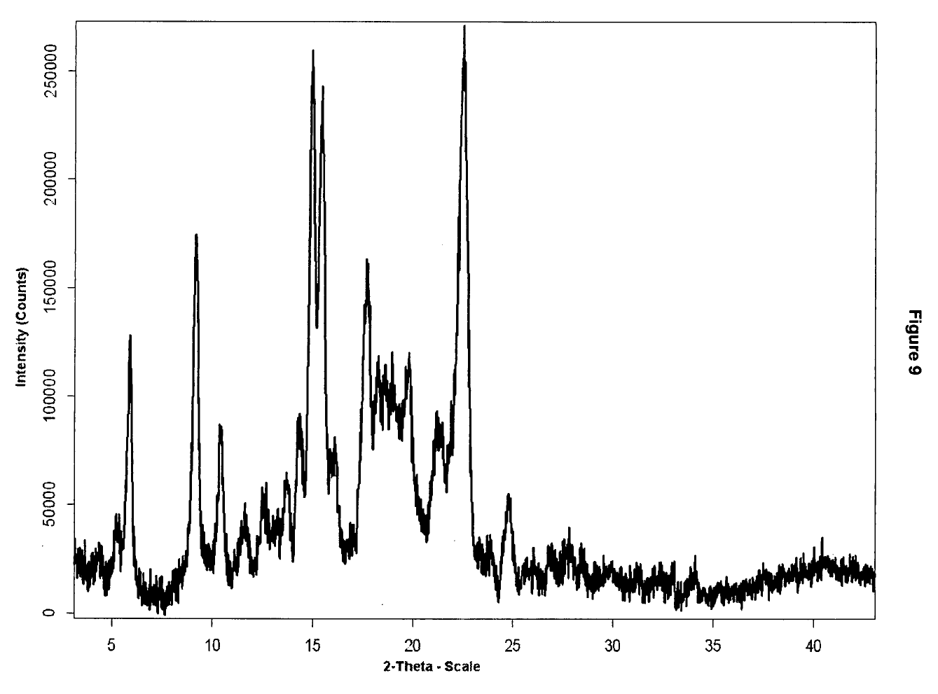

### DETAILED DESCRIPTION OF THE INVENTION

As used herein the term “amorphous” relates to solid material which lacks a regular crystalline structure.

The term “room temperature” as used herein indicates that the applied temperature is not critical and that no exact temperature value has to be kept. Usually, “room temperature” is understood to mean temperatures of about 15° C. to about 25° C. (see e.g. EU Pharmacopoeia 5.0, page 6).

The inventors of the present invention have identified novel polymorphs of Tigecycline. The novel polymorphs have distinct physical properties and may be characterized e.g. by a typical X-ray powder diffraction pattern, infrared spectrum or a characteristic differential scanning calorimetric (DSC) curve. Each of these characteristics on its own is sufficient to unambiguously define and identify the new polymorphs but they also may be combined with each other.

The present invention relates to a novel form VIII of Tigecycline. Form VIII of Tigecycline is an acetone solvate, hereinafter referred to as “form VIII” characterized by an X-ray powder diffraction pattern with peaks at 2-theta angles of 5.1°±0.2°, 9.1°±0.2°, 10.4°±0.2°, 12.8°±0.2°, 13.8°±0.2°, 14.9°±0.2°, 15.4°±0.2°, 16.4°±0.2°, 17.1°±0.2°, 18.6°±0.2°, 20.4°±0.2°, 21.8°±0.2°, 24.2°±0.2° and 25.8°±0.2°.

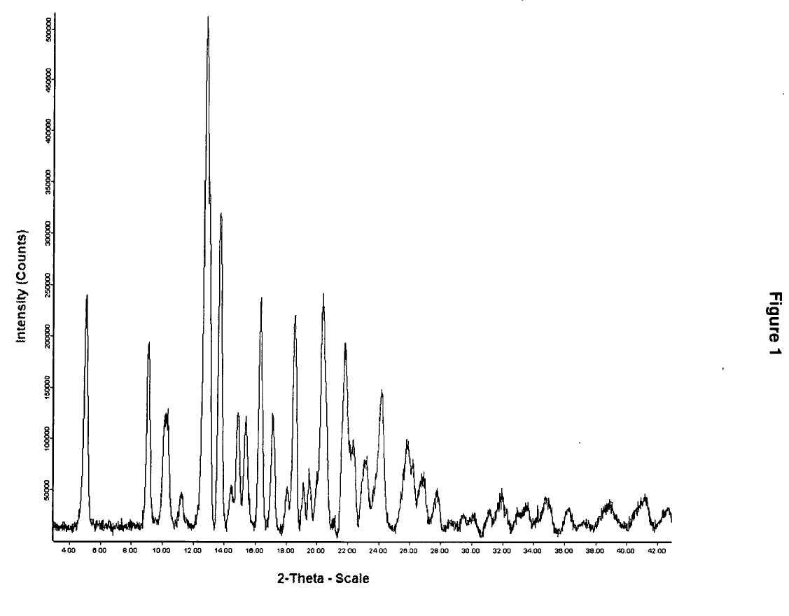

In another aspect the present invention relates to a novel form VIII of Tigecycline characterized by an X-ray powder diffraction pattern substantially in accordance with Table 1 and FIG. 1. The X-ray powder diffraction pattern of form VIII clearly can be distinguished from those of forms I-V from patent number WO 2006/128150 and also from form VI disclosed in this patent.

TABLE 1
X-Ray Powder Diffraction (XRPD) pattern
of form VIII of Tigecycline

| Angle | relative Intensity |
|---|---|
| [°2-theta] | [%] |
| 5.1 | 47.1 |
| 9.1 | 38.2 |
| 10.4 | 25.3 |
| 12.8 | 100.0 |
| 13.8 | 62.6 |
| 14.9 | 24.6 |
| 15.4 | 23.8 |
| 16.4 | 46.8 |
| 17.1 | 24.4 |
| 18.6 | 43.4 |
| 20.4 | 47.5 |
| 21.8 | 38.1 |
| 24.2 | 29.1 |
| 25.8 | 19.8 |

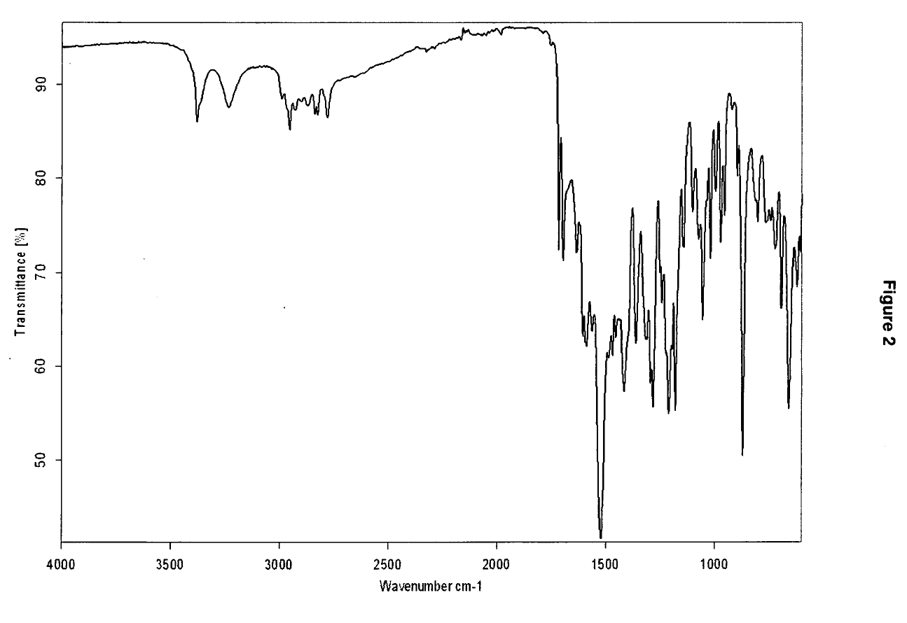

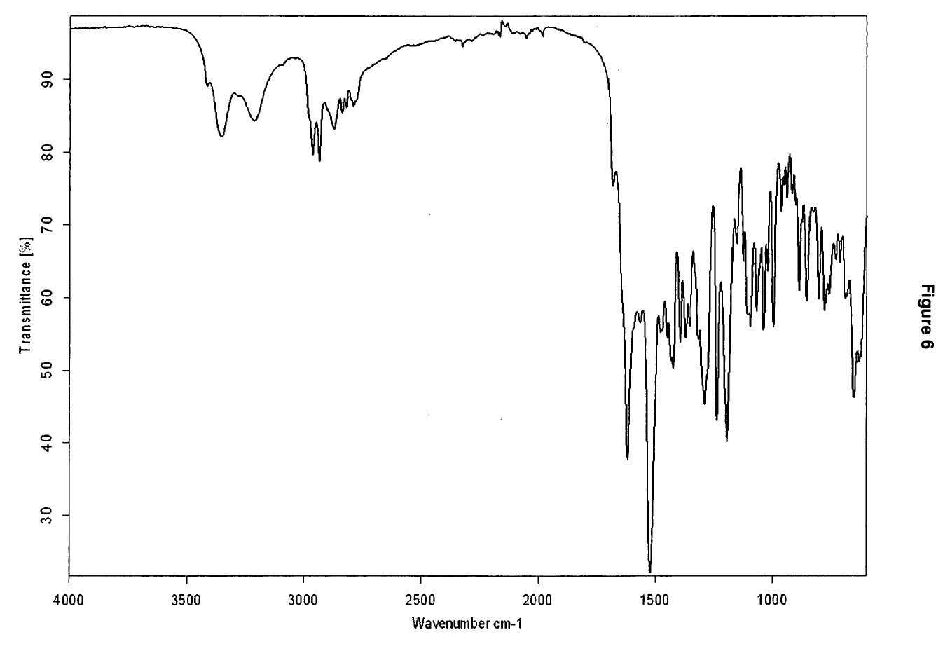

Form VIII of Tigecycline may also be characterized by a typical infrared spectrum as shown in FIG. 2. Accordingly, in a further preferred embodiment, the present invention relates to form VIII of Tigecycline characterized by an infrared spectrum substantially in accordance with FIG. 6. Characteristic bands are present at 3381±2 cm⁻¹, 3231±2 cm⁻¹, 2953±2 cm⁻¹, 1716±2 cm⁻¹, 1695±2 cm⁻¹, 1520±2 cm⁻¹, 1415±2 cm⁻¹, 1210±2 cm⁻¹, 1073±2 cm⁻¹, 870±2 cm⁻¹, 693±2 cm⁻¹, and 659±2 cm⁻¹.

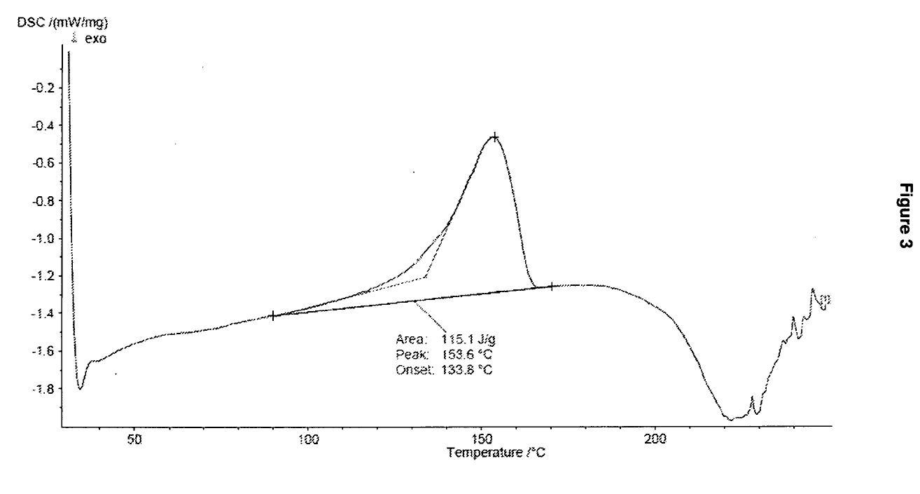

In addition, form VIII of Tigecycline shows a typical DSC curve at a heating rate of 10° C./min. The DSC curve in FIG. 3 displays a broad endothermic peak which is due to desolvation and melting.

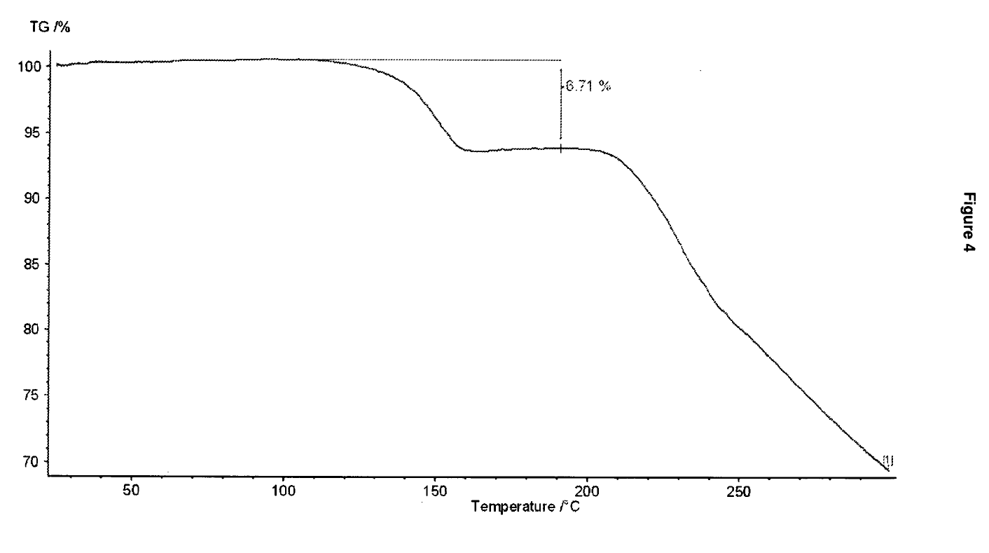

FIG. 4 shows the TGA curve of form VIII of Tigecycline, which displays a total weight loss of about 6.7%, due to the desolvation process. However the acetone content in the crystal lattice may vary. The inventors found batches with 6.7% and 9.3% determined by TGA and different batches with 8.4% and 8.6% determined by GC. Therefore Form VIII of Tigecycline of the present invention tends to be an acetone monosolvate.

In one embodiment the present invention provides a first process of preparing form VIII of Tigecycline, comprising the steps of:
a) slurrying Tigecycline in acetone at 30° C. or below;
b) stirring the slurry at 30° C. or below to effect transformation of the suspended form of Tigecycline into form VIII; and
c) isolating crystalline form VIII of Tigecycline;

In step a) Tigecycline is preferably slurried at a concentration of 30 to 500 g/L, more preferably 50 to 200 g/L, most preferably 100 to 150 g/L.

In step b) the temperature at which the suspension is stirred in order to effect transformation of the suspended form of Tigecycline into form VIII depends on the form and concentration of Tigecycline used and on the solvent used, but usually it will be in the range from 0 to 30° C. However, it is crucial that the temperature is chosen such that the used form of Tigecycline remains in the condition of a suspension and does not become dissolved. Optionally, the method can further comprise seeding the slurry with Tigecycline form VIII.

Furthermore the present invention provides a second process of preparing form VIII of Tigecycline, comprising the steps of:
a) slurrying Tigecycline in acetone at 30° C. or below;
b) heating the suspension to 56° C. or below under stirring to obtain a clear solution;
c) slowly cooling down the suspension to 30° C. or below to effect crystallization; and
d) isolating crystalline form VIII of Tigecycline;

The concentration of Tigecycline in step a) preferably ranges from 5 to 40 g/L, more preferably from 10 to 40 g/L, most preferably from 20 to 40 g/L.

The temperature in step b) may be in the range from 40 to 56° C., depending on the form and concentration of Tigecycline used. However the temperature should be chosen such that a clear solution is obtained.

“Slow cooling” as mentioned in step c) means in this special case a decrease in temperature from e.g. the boiling point of acetone to 0 to 5° C. preferably within 1 to 24 hours, more preferably within 2 to 12 hours, most preferably within 3 to 6 hours.

The crystallization step c) of the above process may be facilitated by adding seed crystals of form VIII of Tigecycline.

The processes represent practical methods of purifying Tigecycline, because most of the impurities of Tigecycline are more soluble in acetone and remain in solution. Tigecycline (1.7 total impurities, with a C₄-epimer content of 1.0%) received from synthesis, for example, was recrystallized with acetone to obtain the acetone solvate in high purity (0.4% total impurities, with a C₄-epimer content of 0.1%).

In addition form VIII of Tigecycline is also a particularly suitable form for the isolation of Tigecycline in the last step of the synthesis of Tigecycline. If, for example 9-chloroacetaminominocycline is reacted with tert.-butylamine in dimethylacetamide Tigecycline can be obtained after a simple extractive work up in high yield and in high purity without an additional purification step.

When crystalline form VIII of Tigecycline (GC: 8.6% acetone) was lyophilized, surprisingly no solvent was found anymore (GC: <0.1% acetone). Therefore a product with high purity was obtained.

The inventors of the present invention have found a novel crystalline form VIII of Tigecycline with suitable physical and chemical properties, such as stability, hygroscopicity, purity and solubility, for pharmaceutical production.

A suitable crystalline form of Tigecycline for formulating an anti-infective medicament first of all is required to be thermodynamic stable, in order to avoid formation of degradation products. Hence the different crystalline forms of Tigecycline were stored for 7 days at 80° C. Table 4 displays the total impurity and 4-Epi-Tigecycline content after storing at the above mentioned conditions. Form I and form II of WO 2006/128150 show a tremendous increase in both, total impurities and 4-Epi-Tigecycline content, and are as a consequent to instable to be used in a formulation process. On the other hand form VIII shows adequate stability data.

Furthermore suitable crystalline forms of Tigecycline should be low hygroscopic, as water uptake may cause the formation of undesired byproducts like e.g. 4-Epi-Tigecycline. Table 5 displays the water uptake of the different crystalline forms of Tigecycline after open storage for 24 hours at 80% relative humidity. Form III of WO 2006/128150 shows a water uptake of 7.32% which is not acceptable, hence form III of WO 2006/128150 is no suitable form for the formulation of an anti-infective medicament. However, form VIII practically shows no water uptake at all.

During the formulation process Tigecycline undergoes a lyophilization process, where the active substance is dissolved in water before lyophilizing. Hence crystalline forms of Tigecycline are required to show suitable water solubility. As displayed in Table 6 form I and form II of WO 2007/127292 clearly show the worst water solubility of all forms and are therefore not the first choice for the lyophilizing step. Although form VIII of the present invention does not show the highest solubility, the value is appropriate for lyophilizing.

In addition, when crystalline form VIII of Tigecycline (GC: 8.6% acetone) was lyophilized, surprisingly no solvent was found anymore (GC: <0.1% acetone). Therefore a product with high purity was obtained.

Moreover crystalline form VIII of Tigecycline is straight forward to prepare and obtained in pure crystalline form by the processes described above, in contrast to form IV of WO 2006/128150.

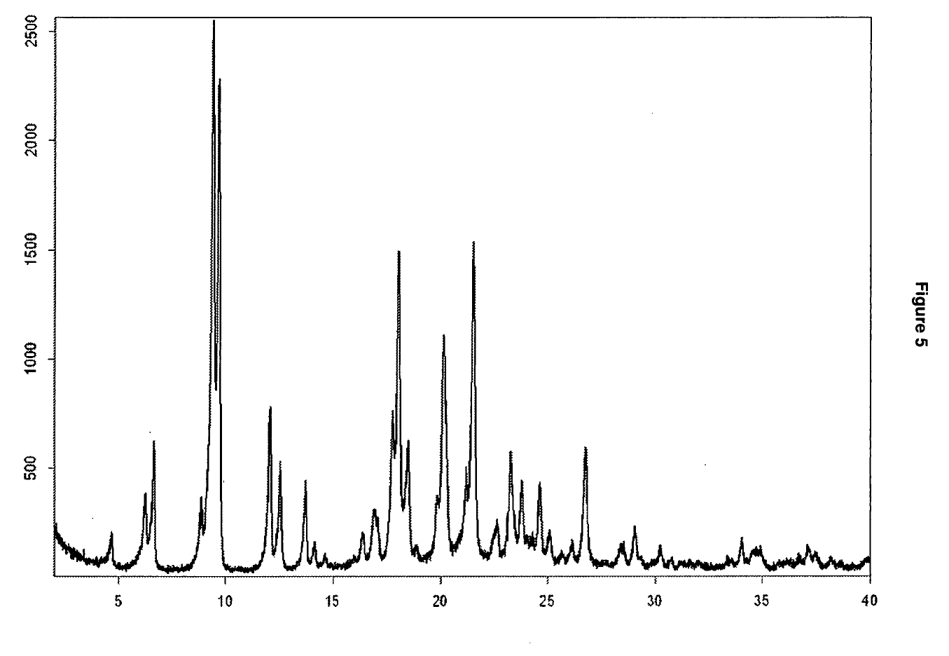

The present invention also relates to a novel form VI of Tigecycline characterized by an X-ray powder diffraction pattern with peaks as shown in table 1 at 6.3, 6.7, 8.9, 9.4, 9.7, 12.1, 12.5, 13.7, 17.0, 17.8, 18.0, 18.5, 20.1, 21.5, 22.6, 23.3, 23.8 and 24.6 degrees 2 theta±0.2°. A characteristic X-ray powder diffraction pattern of form VI of Tigecycline is shown in FIG. 5 and some characteristic peaks are listed in Table 2.

Accordingly, in a preferred embodiment, the present invention relates to a novel form VI of Tigecycline characterized by an X-ray powder diffraction pattern substantially in accordance with Table 2 and FIG. 5.

Form VI possesses peaks at 6.3° 2θ and at 6.7° 2θ. None of the above mentioned forms I to V shows peaks at these positions. Form III of patent number WO 2006/128150 for example possesses a peak at position 6.0° 2θ, which is significantly different from 6.7° 2θ. As a result the found form can be seen as unique and new crystalline polymorphic form VI. In identifying and characterizing form VI, one may also rely on some or all of the other peaks from the X-ray powder diffraction pattern of form VI in FIG. 5.

Form VI of Tigecycline may be also characterized by a typical infrared spectrum as shown in FIG. 6. Accordingly, in a further preferred embodiment, the present invention relates to form VI of Tigecycline characterized by an infrared spectrum substantially in accordance with FIG. 6. Characteristic bands are present at 3353, 3213, 1617, 1522, 1236, 1194, 1067, 1038, 887, 855, 779 and 654 cm-1.

TABLE 2
X-Ray Powder Diffraction (XRPD) pattern
of form VI of Tigecycline.

| Angle | relative Intensity |
|---|---|
| 2-Theta | [%] |
| 6.3 | 16 |
| 6.7 | 24 |
| 8.9 | 15 |
| 9.4 | 100 |
| 9.7 | 85 |
| 12.1 | 36 |
| 12.5 | 25 |
| 13.7 | 16 |
| 17.0 | 10 |
| 17.8 | 27 |
| 18.0 | 51 |
| 18.5 | 24 |
| 20.1 | 39 |
| 21.5 | 55 |
| 22.6 | 10 |
| 23.3 | 20 |
| 23.8 | 16 |
| 24.6 | 18 |

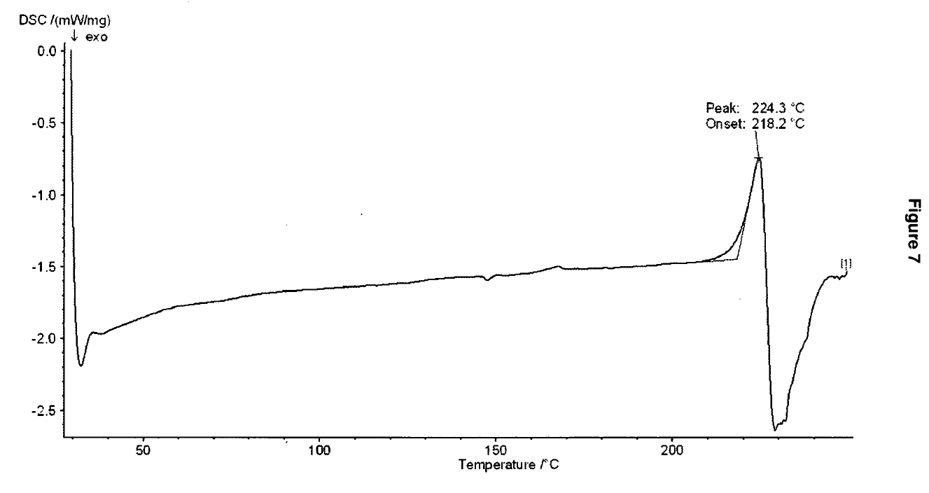

In addition, form VI of Tigecycline shows a typical DSC curve at a heating rate of 10° K/min. It can be seen in FIG. 7 that the DSC curve of form VI shows an endothermic peaks with onset temperature of about 217° C. Melting points do not necessary identify different polymorphs. For example melting point onsets of Form I-V range from 167° C.-174° C. This small range does not allow to differ between polymorphic forms. However form VI shows a melting point onset at about 217° C. which is significantly different from the other polymorphic forms. In this case it's possible to distinguish between Form VI and the other known forms.

Form VI of Tigecycline is an anhydrous form, hereinafter also referred to as “form VI”, which is more thermodynamically stable than the previously known polymorphic form of Tigecycline and hence is suitable for bulk preparation and handling. Form VI of Tigecycline has been found to be of low hygroscopicity and does not substantially convert into a hydrated form of Tigecycline.

In one embodiment, the present invention provides a first process for preparing form VI of Tigecycline, comprising the steps of:

a) dissolving Tigecycline in a suitable solvent
b) stirring the solution at room temperature or below to effect crystallization
c) optionally isolating crystalline form VI of Tigecycline

For preparing form VI of Tigecycline according to the above first process, any other form of Tigecycline may be used, e.g. the amorphous form or crystalline form I to V disclosed in WO 2006/128150. In addition, also forms of low crystalline or mixtures of two or more different forms of Tigecycline.

According to the above first process a suitable solvent in step a) is acetone.

Regarding the preparation of form VI and form VIII with acetone as a solvent, the discriminating features are listed in the following table:

TABLE 3

| Preparation of Form VIII | Preparation of Form VI |
|---|---|
| Slurry in acetone at room temperature | Slurry in acetone at room |
| | temperature plus seed crystals |
| | of form VI |
| Solution in acetone at elevated | Solution in acetone at room |
| temperature and crystallization at | temperature and crystallization |
| elevated temperature (e.g. >30° C.) | at room temperature |

In another embodiment, the present invention provides a second process for preparing form VI of Tigecycline characterized in that a suspension of Tigecycline in a suitable solvent is seeded with crystals of form VI and the suspension is stirred at a suitable temperature in order to effect transformation of the used form of Tigecycline into form VI.

Just as for the first process described above also for this second process for preparing form VI of Tigecycline any other form of Tigecycline, i.e. any crystalline unsolvated or solvated form, non-crystalline or amorphous form may be used.

However, this second process is based on the solution mediated transformation of any form of Tigecycline which is thermodynamically less stable under given conditions than form VI. Therefore, the transformation into form VI is a thermodynamically controlled process due to the fact that form VI has the lowest Gibbs free energy. Consequently, all known solid crystalline or non-crystalline forms of Tigecycline may be used in the present process.

According to the above second process a suitable solvent is a solvent or solvent mixture which does not form a crystalline solvate with Tigecycline and in which the substance is not highly soluble. In a preferred embodiment, the solvent used in the above second process for preparing form VI of Tigecycline is selected from acetone and acetonitrile.

The temperature at which the suspension is stirred in order to effect transformation of the suspended form of Tigecycline into form VI depends on the form of Tigecycline and the solvent used. Room temperature or an elevated temperature may be applied but usually it will be in the range of 10° C. to 40° C. However, it is crucial that solvent and temperature are chosen such that the used form of Tigecycline remains in the condition of a suspension and does not become dissolved. It is well within the general knowledge of a person skilled in the art to determine temperature accordingly.

In addition the present invention provides another process of preparing form VI comprising the steps of:
a) dissolving Tigecycline in nitromethane
b) stirring the solution at room temperature or lower to effect crystallization
c) isolating crystalline form VI of Tigecycline

Nitromethane must be used only in low concentrations (<0.5 mg/day). It is a suitable solvent for the purification of Tigecycline because most impurities are soluble in nitromethane and remain in solution. Further higher yields of the product are reached by using nitromethane instead of acetone.

The crystallization step b) of the above process may be facilitated by adding seed crystals of form VI of Tigecycline. Accordingly, in a preferred embodiment, in the above process in step b) seed crystals of form VI of Tigecycline are added

The inventors of the present invention found novel crystalline forms of Tigecycline, namely forms VIII and VI, with suitable properties for the preparation of an anti-infective medicament.

After storing the different crystalline forms of Tigecycline for 7 days at 80° C., forms VIII and VI of the present invention clearly show higher stability than e.g. form I and form II of WO 2006/128150 which is displayed in Table 4. Form I and form II of WO 2006/128150 show a tremendous increase in both, total impurities and 4-Epi-Tigecycline content, and are consequently to instable to be used in a formulation process. On the other hand forms VIII and VI show adequate stability data.

Furthermore suitable crystalline forms of Tigecycline should be of low hygroscopicity, as water uptake may cause the formation of undesired byproducts like e.g. 4-Epi-Tigecycline. Table 5 displays the water uptake of the different crystalline forms of Tigecycline after open storage for 24 hours at 80% relative humidity. Form III of WO 2006/128150 shows a water uptake of 7.32% which is not acceptable, hence form III of WO 2006/128150 is no suitable form for the formulation of an anti-infective medicament. However, 0.11% water are taken up by form VIII, which is a very low value and therefore appropriate for the formulation process.

During the formulation process Tigecycline undergoes a lyophilization process, where the active substance is dissolved in water before lyophilizing. Hence crystalline forms of Tigecycline are required to show suitable water solubility. As displayed in Table 6, form VIII of the present invention does not show the highest solubility, but the value is appropriate for lyophilizing.

TABLE 4
Stability data after storing different forms
of Tigecycline at 80° C. for 7 days

| | 4-Epi- | 4-Epi- | Total | Total |
|---|---|---|---|---|
| | Tigecycline | Tigecycline | impurities | impurities |
| | ambient | 7 days at | ambient | 7 days at |
| Form | conditions* | 80° C. | conditions* | 80° C. |
| WO 2006/128150 | | | | |
| I | 0.36 | 2.15 | 0.75 | 11.20 |
| II | 1.04 | 10.83 | 1.60 | 18.44 |
| III | 0.16 | 2.18 | 0.16 | 4.46 |
| IV | 0.23 | 0.38 | 0.40 | 0.87 |
| V | <0.05 | 0.18 | 0.22 | 0.37 |
| Present invention | | | | |
| VI | 0.17 | 0.38 | 0.24 | 0.91 |
| VIII | 0.15 | 0.70 | 0.43 | 2.13 |

*4-Epi-Tigecycline and Total impurity content at ambient conditions depend amongst others on the purity of the starting material

TABLE 5
Water content and hygroscopicity data
of different forms of Tigecycline

| | | water | water |
|---|---|---|---|
| | water content [%] | content | uptake |
| | ambient | 1 day at | 1 day at |
| Form | conditions | 80% rH | 80% rH |
| WO 2006/128150 | | | |
| I | 2.01 | 5.01 | 3.00 |
| II | 2.93 | 10.04 | 7.11 |
| III | 0.69 | 8.01 | 7.32 |
| IV | 2.90 | 3.33 | 0.43 |
| V | 0.19 | 1.88 | 1.69 |
| Present invention | | | |
| VI | 0.18 | 0.25 | 0.07 |
| VIII | 0.10 | 0.21 | 0.11 |

TABLE 6
Solubility data of different forms of Tigecycline

| Form | Concentration [mg/ml] |
|---|---|
| WO 2006/128150 | |
| I | 185 |
| II | 177 |
| III | 174 |
| IV | 183 |
| V | 134 |
| Present invention | |
| VI | 76 |
| VIII | 141 |

In another aspect the invention provides a new process for preparing form I comprising the steps of:

a) dissolving Tigecycline in ethanol,
b) stirring the solution at room temperature or below to effect crystallization
c) isolating crystalline form I of Tigecycline

For preparing form I of Tigecycline according to the above process, any other form of Tigecycline may be used, e.g. the amorphous form and also forms of low crystallinity or mixtures of two or more different forms of Tigecycline. The crystallization step b) of the above process may be facilitated by adding seed crystals of form I of Tigecycline. Ethanol as solvent is more suitable to get form I than a mixture of acetone and methanol as mentioned in WO 2006/128150 and the process represents a practical method for purification of Tigecycline, because most of the impurities of Tigecycline are more soluble in ethanol and remain in solution.

In another aspect the invention provides a new process for preparing form III comprising the steps of:

a) dissolving Tigecycline in methyl ethyl ketone,
b) stirring the solution at room temperature or below to effect crystallization
c) isolating crystalline form III of Tigecycline

For preparing form III of Tigecycline according to the above process, any other form of Tigecycline may be used, e.g. the amorphous form and also forms of low crystallinity or mixtures of two or more different forms of Tigecycline. The crystallization step b) of the above process may be facilitated by adding seed crystals of form III of Tigecycline.

Surprisingly it has been found, that the process represents a practical method to get form III in high polymorphic purity. WO 2006/128150 discloses the preparation of form III by crystallizing Tigecycline out of dichloromethane or by slurrying Form I in dichloromethane. The comparison of the peak positions of the diffractogram of form III crystallized according to the conditions described here, with the positions listed for the diffractogram of form III in WO 06/128150 reveals good correspondence. No correspondence is found for two reflections at 8.294 and 13.132, listed for form III in WO 06/128150 (see table 7). However, since form I has an intense reflection at 8.292 and also at 13.167, according to the peak positions listed for form I in WO 06/128150, it is concluded that form III, described in WO 06/128150, also contained form I. Therefore form III, crystallized according to the conditions described here, is essentially pure form III.

The present invention includes the essentially pure Form III with no peak at position 8.3° 2θ.

TABLE 7
Comparison Form III pure with Form III from WO 06/128150

| Form III pure | Form III (WO 06/128150) |
|---|---|
| 5.2 | 5.229 |
| 5.9 | 5.95 |
| — | 8.294 |
| 9.1 | 9.319 |
| 10.4 | 10.551 |
| 11.6 | 11.834 |
| — | 13.132 |
| 13.7 | 13.69 |
| 14.4 | 14.384 |
| 14.9 | 14.951 |
| 15.4 | 15.505 |
| 17.7 | 17.758 |
| 21.2 | 21.376 |
| 24.8 | 24.773 |

The novel form VI of Tigecycline of the present invention may be used alone as antibacterial drug or in the form of a suitable pharmaceutical composition containing the novel form. Accordingly, the present invention relates to form VI of Tigecycline for use as a medicament.

The novel form VI of Tigecycline is particularly useful for the treatment of infections. Therefore, the present invention also relates to the use of form VI of Tigecycline for the preparation of a medicament for the treatment of infections.

The present invention further relates to a pharmaceutical composition for parenteral use comprising an effective amount of form VI of Tigecycline.

The invention is further described by reference to the following examples. These examples are provided for illustration purposes only and are not intended to be limiting the present invention in any way.

### EXAMPLES

Infrared spectra were collected on a diamond ATR cell with an Bruker Tensor 27 FTIR spectrometer with 4 cm-1 resolution.

Powder diffractograms of form I and VI were collected on an AXS-BRUKER X-ray powder diffractometer D-8 with an E-dispersive counter in parallel beam optics using the following acquisition conditions: tube anode: Cu, 40 kV, 40 mA; continuous scan 2-40° theta/2theta, step size 0.01°, counting time 2 seconds per step, room conditions

Powder diffractograms of form III and VIII were collected on a Unisantis XMD 300 X-ray powder diffractometer with a position sensitive detector in parallel beam optics using the following acquisition conditions: tube anode: Cu , 40 kV, 0.8 mA; 3-43° theta/2theta; simultaneous detection of regions of 10° per step with detector resolution 1024, counting time 300 seconds per step. Samples were measured in a standard plastic sample holder on a rotating sample spinner. Since the sample holder material displays a diffraction peak at approximately 22.4 degrees 2-theta in the diffractograms, peaks at this position are not listed as characteristic peaks.

Differential scanning calorimetry (DSC) was performed on a Netzsch DSC 204. Samples were heated in 25 μl Al-Pans with loose lids from room temperature to 250° C. at a rate of 10° C./min. Nitrogen (purge rate 20 ml/min) was used as purge gas.

Thermogravimetric analysis (TGA) was performed on a Netzsch STA 409 PC/PG instrument. Samples were heated in an Al₂O₃ crucible from room temperature to 300° C. at a rate of 10° C./min. Nitrogen (purge rate 50 ml/min) was used as purge gas.

### Example 1

### Preparation of Form VIII

105.3 mg Tigecycline (form III) were slurried in 1.5 ml acetone at room temperature for 15 hours. The solid was filtered off, washed with acetone and dried under vacuum at room temperature for 3 hours to obtain 87.9 mg (83% yield, acetone content not considered) of crystalline form VIII of Tigecycline.

### Example 2

### Preparation of Form VIII

To 200.0 mg Tigecycline (form I) 2 ml acetone were slowly added. The resulting suspension was stirred at room temperature for 21.75 hours before the solid was filtered off, washed with acetone and dried under vacuum at room temperature for about 4 hours to obtain 188.4 mg (94% yield, acetone content not considered) of crystalline form VIII of Tigecycline (HPLC: 99.42%).

### Example 3

### Preparation of Form VIII

A solution of 200.0 mg Tigecycline in 2.5 ml acetone was stirred at 40° C. Within a minute a precipitate appeared and the suspension was stirred for 35 minutes at 40° C. The solid was filtered off, washed with acetone and dried under vacuum for 20.5 hours to obtain 125.5 mg (63% yield, acetone content not considered) of crystalline form VIII of Tigecycline.

### Example 4

### Preparation of Form VIII

To 380.9 mg Tigecycline (form II) 3 ml acetone were slowly added. The resulting suspension was stirred at room temperature for 22.75 hours. The solid was filtered off, washed with acetone and dried under vacuum at room temperature to obtain 362.7 mg (86% yield without solvent) of crystalline form VIII of Tigecycline.

### Example 5

### Preparation of Form VIII

1000.0 mg Tigecycline were slurried in 35 ml acetone at room temperature. The suspension was refluxed (60° C. bath temperature) to obtain a clear solution. The solution was allowed to cool down slowly (within 270 minutes) to room temperature while gently stirring. Then the oil bath was replaced by an ice/water bath to effect complete crystallization. Finally the solid was filtered off, washed with acetone and dried under vacuum at room temperature to obtain 529.5 mg (53% yield, acetone content not considered) of crystalline form VIII of Tigecycline.

### Example 6

### Preparation of form VI

50 mg of amorphous Tigecycline are slurried in 1000 μl acetone at 25° C. to form a suspension. The suspension is stirred for 2 hours at 25° C. The slurry is filtered and the solid washed with acetone. The solid is dried under vacuum at 25° C. for 3 hours to give 30.4 mg (61% yield) of the crystalline form VI.

Melting point: 218° C.

The form VI crystals of Tigecycline obtained above provide an infrared spectrum with peaks at 3353, 3213, 1617, 1522, 1236, 1194, 1067, 1038, 887, 855, 779 and 654 cm-1 (FIG. 6).

The XRPD pattern of form VI of Tigecycline with characteristic XRPD angles and relative intensities is shown in Table 2 and in FIG. 5.

### Example 7

### Preparation of Form VI

50 mg of amorphous Tigecycline are slurried in 667 μl acetonitrile at 25° C. to form a suspension. 2.5 mg of Form VI are added as a seed and the suspension is stirred for 20 minutes at 25° C. The slurry is filtered and the solid washed with acetonitrile. The solid is dried under vacuum at 25° C. for 4 hours to give 40.6 mg (81% yield) of the crystalline form VI.

### Example 8

### Preparation of Form VI

100.0 mg amorphous Tigecycline are dissolved in 1.5 ml acetone at room temperature. After a few seconds stirring at room temperature a yellow precipitate is obtained. The suspension is stirred for 3 hours before the solid is filtered off, washed with acetone and dried at room temperature under vacuum to obtain 63.5 mg (64% yield) of crystalline Tigecycline form VI.

### Example 9

### Preparation of Form VI

50.0 mg amorphous Tigecycline are slurried in 0.6 ml acetone at room temperature. After the addition of seed crystals of Tigecycline form VI the suspension is stirred for 1 hour. The solid is filtered off, washed with acetone and dried at room temperature under vacuum to obtain 35.7 mg (71% yield) of crystalline Tigecycline form VI.

### Example 10

### Preparation of Form VI

39.4 mg of Tigecycline form V are slurried in 800 μl acetone at 25° C. to form a suspension. 2 mg of Form VI are added as a seed and the suspension is stirred at 25° C. for 23 hours. The suspension is filtered and washed with acetone. The solid is dried under vacuum at 25° C. for 4.5 hours to give 18.0 mg (46% yield) of the crystalline form VI.

### Example 11

### Preparation of Form VI

35.1 mg of Tigecycline form IV are slurried in 700 μl acetone at 25° C. to form a suspension. 1.8 mg of form VI are added as a seed and the suspension is stirred at 25° C. for 16 hours. The suspension is filtered and washed with acetone. The solid is dried under vacuum at 25° C. for 5 hours to give 21.6 mg (61% yield) of the crystalline form VI.

### Example 12

### Preparation of Form VI

50.0 mg of amorphous Tigecycline are dissolved in 500 μl nitromethane and stirred at room temperature. After about 1 minute a yellow precipitate is obtained and the suspension is stirred for another 30 minutes at room temperature. The solid is filtered off, washed with nitromethane and dried under vacuum at room temperature for 20 hours to give 42.4 mg (85% yield) of the crystalline form VI.

### Example 13

### Preparation of Form III

To 50 mg of amorphous tigecycline 500 μl of methyl ethyl ketone are added and the clear solution is stirred at 25° C. After about one minute an orange slurry is received which is filtered after 2.5 hours stirring. The solid is dried under vacuum at 25° C. for 21.5 hours to give 37.9 mg (76% yield) of the crystalline Form III.

### Example 14

### Preparation of Form I

To 50 mg of amorphous tigecycline 1 ml of ethanol is added and the clear solution is stirred at 25° C. After some seconds a red slurry is received which is filtered after 30 min stirring. The solid is dried under vacuum at 25° C. for 16 hours to give 34.9 mg (70% yield) of the crystalline Form I.

### Example 15

### Water Solubility Testing

The water solubility determination of the different crystalline forms of Tigecycline was performed on a Perkin Elmer® Lambda 35 UV/VIS spectrometer. The software used was Perkin Elmer® UV WlnLab-5.1. The calibration curve was determined with form II of Tigecycline from WO 2007/127792.

200 mg of Tigecycline were stirred with 1 ml distilled water at room temperature for 30 minutes. The suspension was filtered through a 0.45 μm filter and 100 μl of the solution were transferred into a 1 L volumetric flask. After filling up to the check mark with distilled water the solution was measured at 347 nm.

1. Form VIII of Tigecycline characterized by an X-ray powder diffraction pattern substantially in accordance with Table 2 and FIG. 5.

2. Form VIII of Tigecycline of claim 10 characterized by an infrared spectrum substantially in accordance with FIG. 6.

3. Form VIII of Tigecycline of claim 10 characterized by a DSC curve substantially in accordance with FIG. 7.

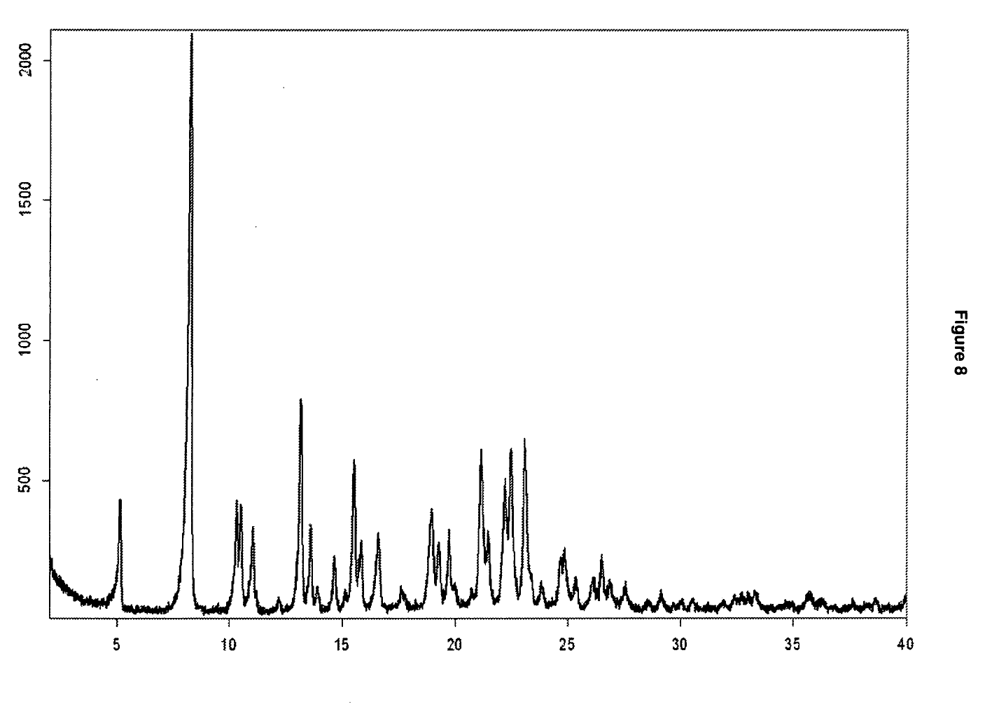

4. Form VIII of Tigecycline of claim 10 characterized by TGA curve substantially in accordance with FIG. 8.

5. A process of preparing form VIII of Tigecycline comprising the steps of:
a) slurrying Tigecycline in acetone at 30° C. or below;
b) stirring the suspension at 30° C. or below to effect transformation of the used form of Tigecycline into form VIII; and
c) isolating crystalline form VIII of Tigecycline;

6. A process of preparing form VIII of Tigecycline, comprising steps of:
a) slurrying Tigecycline in acetone at 30° C. or below;
b) heating the suspension to 56° C. or below under stirring to obtain a clear solution;
c) slowly cooling down the solution to 25 to 0° C. to effect crystallization; and
d) isolating crystalline form VIII of Tigecycline;

7. Form VIII of Tigecycline for use as medicament.

8. Use of form VIII of Tigecycline for the preparation of a medicament for the treatment of infections.

9. Pharmaceutical composition comprising an effective amount of form VIII of Tigecycline.

10. Form VI of Tigecycline characterized by an X-ray powder diffraction pattern with peaks at 6.3 and 6.7 degrees 2θ.

11. Form VI of Tigecycline of claim 1 characterized by an X-ray powder diffraction pattern substantially in accordance with Table 1 and FIG. 1.

12. Form VI of Tigecycline of claim 1 characterized by an infrared spectrum substantially in accordance with FIG. 2.

13. Form VI of Tigecycline of claim 1 characterized by an endothermic peak with onset temperature of about 217° C. at a heating rate of 5° K/min in differential scanning calorimetry.

14. Process for preparing form VI of Tigecycline, comprising the steps of:
a) dissolving Tigecycline in a suitable solvent
b) stirring the solution at room temperature or below to effect crystallization
c) optionally isolating crystalline form VI of Tigecycline

15. Process according to claim 5, wherein the solvent in step a) is acetone.

16. Process according to claim 5, wherein the solvent in step a) is nitromethane.

17. Process for preparing form VI of Tigecycline characterized in that a suspension of Tigecycline in a suitable solvent is seeded with crystals of form VI and the suspension is stirred at a suitable temperature in order to effect transformation of the used form of Tigecycline into form VI

18. Form VI of Tigecycline for use as medicament.

19. Use of form VI of Tigecycline for the preparation of a medicament for the treatment of infections.

20. Pharmaceutical composition comprising an effective amount of form VI of Tigecycline.

21. Process for preparing form I of Tigecycline comprising the steps of:
a) dissolving Tigecycline in ethanol,
b) stirring the solution at room temperature or below to effect crystallization
c) isolating crystalline form I of Tigecycline

22. Form III of Tigecycline in essentially pure polymorphic form.

23. Essentially pure polymorphic form III of claim 9 with no peak at position 8.3° 2θ.

24. Process for preparing form III in essentially pure form comprising the steps of:
a) dissolving Tigecycline in methyl ethyl ketone,
b) stirring the solution at room temperature or below to effect crystallization
c) isolating crystalline form III of Tigecycline
